(19) United States
(12) Patent Application Publication
(10) Pub. No.: US 2014/0072573 A1
(43) Pub. Date: Mar. 13, 2014

(54) METHOD OF TREATING RHEUMATOID ARTHRITIS

(71) Applicant: Institut National de la Sante et de la Recherche Medicale (INSERM), Paris (FR)

(72) Inventors: Isabelle Vilgrain, Sassenage (FR); Adama Sidibe, Saint Martin d'Heres (FR); Tiphaine Mannic, Cervens (FR); Olivier Vittecoq, Saint Ouen de Thouberville (FR); Xavier Le Loet, Rouen (FR)

(73) Assignee: Institut National de la Sante et de la Recherche Medicale (INSERM), Paris (FR)

(21) Appl. No.: 14/018,879

(22) Filed: Sep. 5, 2013

Related U.S. Application Data
(60) Provisional application No. 61/698,222, filed on Sep. 7, 2012.

(51) Int. Cl. G01N 33/68

(52) U.S. Cl. CPC G01N 33/6872
USPC 424/158.1; 435/7.92; 514/7.6; 514/313; 514/249

(57) ABSTRACT

The invention relates to a method of treating rheumatoid arthritis in a patient comprising the steps of:

a) providing a biological sample from a patient,

b) measuring the level of soluble VE-cadherin in the biological sample obtained at step a);

c) comparing said level of soluble VE-cadherin with a predetermined reference value, and

if the level of soluble VE-cadherin measured at step b) is higher that the predetermined reference value,

treating the patient until a basal level of soluble VE-cadherin is reached.

### FIELD OF THE INVENTION

The invention relates to a method of treating rheumatoid arthritis in a patient.

### BACKGROUND OF THE INVENTION

Rheumatoid arthritis (RA) is a systematic inflammatory autoimmune disorder that affects up to 1% of the European population. RA is characterized by irreversible joint damages, whit disability and ultimately accelerated atherosclerotic cardiovascular and coronary heart disease. Chronic infiltration of the joints by activated immune competent cells including macrophages, T and B cells, together with synovial tissue hyperplasia, leads to cartilage and bone destruction after several years. Although the causes of RA are not fully understood, numerous studies indicate that cytokines are critical in the processes that cause inflammation and joint destruction, TNF-alpha being definitively the prominent one. Currently, in clinic, if diseases activity cannot be controlled with conventional disease modifying anti-rheumatic drugs (DMARD), anti-TNF biotherapies are used. Although a major breakthrough has emerged in the management of RA patients with TNF-alpha blockade, it is not curative and its effects are only partial, non responses common and loss of effect are observed. When patients do not respond to TNF blocking agents (40%), RTX is often prescribed to induce complete remission in the majority of patients.

Taking into account the cost of these treatments, the persisting doubts about potential long term adverse events and the availability of other efficient biotherapies in the treatment of RA, selection, adjustment and monitoring of therapy for a patient is a key issue.

### SUMMARY OF THE INVENTION

In a first aspect, the invention relates to a method of treating rheumatoid arthritis in a patient comprising the steps of:
a) providing a biological sample from a patient,
b) measuring the level of soluble VE-cadherin in the biological sample obtained at step a);
c) comparing said level of soluble VE-cadherin with a predetermined reference value, and

if the level of soluble VE-cadherin measured at step b) is higher that the predetermined reference value,

treating the patient until a basal level of soluble VE-cadherin is reached.

In a second aspect, the invention relates to a method of treating rheumatoid arthritis in a patient comprising the steps of:
a) providing a biological sample from a patient suffering from rheumatoid arthritis;
b) measuring the level of soluble VE-cadherin in the biological sample obtained at step a);
c) administering to the patient an effective amount of a TNF-alpha blocking agent;
d) providing a biological sample from the treated patient at step c);
e) measuring the level of soluble VE-cadherin in the biological sample obtained at step d);
f) comparing the levels of soluble VE-cadherin measured in biological samples at step b) and d), and

if the level of soluble VE-cadherin measured at step d) is lower that the level of soluble VE-measured at step b),

treating the patient by pursuing to administer an effective amount of a TNF-alpha blocking agent until a basal level of soluble VE-cadherin is reached.

### DETAILED DESCRIPTION OF THE INVENTION

The inventors have demonstrated that TNF-alpha induces the shedding of VE-cadherin leading to the generation of soluble VE-cadherin. They have also shown that level of soluble VE-cadherin is detected in sera from the 63 Rheumatoid Arthritis (RA) patients and is positively correlated with the Disease Activity Score at baseline and after 1-year followup. These findings provide the first evidence of VE-cadherin proteolysis upon TNF-alpha stimulation and suggest potential clinical relevance of soluble VE-cadherin in management of RA notably for monitoring the responsiveness of patients to new therapies with TNF-alpha blocking agents (e.g. infliximab, adalimumab or etanercept) as well as stratifying patients.

### Definitions

Throughout the specification, several terms are employed and are defined in the following paragraphs.

As used herein, a “biological sample” refers to a biological sample obtained for the purpose of in vitro evaluation. Typical biological samples to be used in the method according to the invention are blood samples (e.g. whole blood sample or serum sample). In a preferred embodiment, said blood sample is a serum sample obtained from a patient to be tested.

As used herein, the term “patient” refers to a mammal, preferably a human. Typically, a patient has been previously diagnosed rheumatoid arthritis.

As used herein, “measuring” encompasses detecting or quantifying.

As used herein, “detecting” means determining if the soluble VE-cadherin is present or not in the biological sample and “quantifying” means determining the amount of the soluble VE-cadherin in the biological sample.

As used herein, the term “VE-cadherin” has its general meaning in the art and refers to vascular endothelial cadherin. This protein of 784 amino acids is an endothelial-specific cadherin localized at the intercellular junctions of most organs and tissues. By way of example, human VE-cadherin is provided under GenBank accession number CAA56306 and has been described in the international application WO 98/25946. VE-cadherin comprises an extracellular domain, which consists of five cadherin-like repeats, a transmembrane domain and a short cytoplasmic tail as previously described (Corada et al., 2001).

As used herein, the terms “soluble VE-cadherin”, “sVE-cadherin”, “VE-cadherin extracellular domain” or “VE-90” are used interchangeably and refer to the VE-cadherin fragment 1-593 of the sequence available from GenBank under accession number CAA56306 having an apparent molecular weight of about 90 kDa as described in the international patent application WO 2008/062314. The terms “soluble VE-cadherin” may also include subfragments of said VE-cadherin fragment 1-593, such as the subfragment Asp48-Glu478 also described in the international patent application WO 2008/062314.

As used herein, the term “disease modifying antirheumatic drugs (DMARDs)” refers to a molecule, such as protein or small molecule, defined by their use in rheumatoid arthritis to slow down disease progression. The term is often used in contrast to non-steroidal anti-inflammatory drugs (which refer to molecules that treat the inflammation but not the underlying cause) and steroids (which blunt the immune response but are insufficient to slow down the progression of the disease).

Such DMARDs include, but are not limited to, tumor necrosis factor (TNF)-alpha blocking agent, IL-1 receptor antagonists (IL1ra) such as Anakinra (Kineret®), B cell depleting agents such as Rituximab (Rituxan®), anti-IL-6 antibodies (Tocilizumab, Roactemra®), T-cell costimulatory blockers such as Abatacept (Orencia®) as well as other drugs such as for instance methotrexate (MTX), sulfasalazine, leflunomide, antimalarials, gold salts, d-penicillamine, cyclosporin A, cyclophosphamide and azathioprine.

As used herein, the term “TNF-alpha blocking agent” refers to a molecule, such as protein or small molecule that can significantly reduce TNF-alpha properties.

### Methods of Treating Rheumatoid Arthritis According to the Invention

The invention relates to a method of treating rheumatoid arthritis in a patient comprising the steps of:
a) providing a biological sample from a patient,
b) measuring the level of soluble VE-cadherin in the biological sample obtained at step a);
c) comparing said level of soluble VE-cadherin with a predetermined reference value, and

if the level of soluble VE-cadherin measured at step b) is higher that the predetermined reference value,

treating the patient until a basal level of soluble VE-cadherin is reached.

According to the invention, the predetermined reference value may be obtained from a patient, or group of patients, affected with rheumatoid arthritis, in particular from a patient, or group of patients, with non active RA or from a patient, or group of patients, who is not affected with rheumatoid arthritis.

Indeed, the patients who are affected with rheumatoid arthritis are those who have an increased expression of soluble VE-cadherin compared to the patients who are not affected, and among the patients who are affected with rheumatoid arthritis, the expression of soluble VE-cadherin is higher in patients with active RA than in patients with non active RA.

The method of the invention is thus particularly useful to treat a patient with rheumatoid arthritis that is active.

The Rheumatoid Arthritis disease activity can be measured according to the standards recognized in the art, such as the “Disease Activity Score” (DAS) or the American College of Rheumatology (ACR) criteria which are measures of the activity of rheumatoid arthritis. In Europe, the DAS is the recognized standard in research and clinical practice.

The following parameters are included in the calculation (Van Gestel A M, Prevoo M L L, van't Hof M A, et al. Development and validation of the European League Against Rheumatism response criteria for rheumatoid arthritis. Arthritis Rheum 1996; 39:34-40):
Number of joints tender to the touch (TEN)
umber of swollen joints (SW)
Erythrocyte sedimentation rate (ESR)
Patient assessment of disease activity (VAS; mm).

Further, in some embodiments, multiple determinations of level of soluble VE-cadherin over time can be made to facilitate monitoring of treatment. A temporal change in the level of soluble VE-cadherin can be used to predict a clinical outcome, monitor the progression of RA and/or efficacy of appropriate therapies.

In one embodiment, the patient suffering from rheumatoid arthritis does not show an increase of the level of CRP (C-reactive protein) which is used as a marker of inflammation.

According to the invention, the patients who are affected with an active rheumatoid arthritis are those who have an increased expression of soluble VE-cadherin compared to the patients who are affected with a not active rheumatoid arthritis (i.e. the level of soluble VE-cadherin is higher in patients with a high disease activity score (DAS)).

Accordingly, the patient should be treated until a basal level of soluble VE-cadherin is reached. This basal level of soluble VE-cadherin may determined by measuring the level of soluble VE-cadherin in patient, or group of patients, affected with non active rheumatoid arthritis, or from a patient, or group of patients, who is not affected with rheumatoid arthritis.

In one embodiment, the patient is treated with an effective amount of a disease modifying antirheumatic drugs (DMARD).

In a particular embodiment, the patient is treated with an effective amount of a TNF-alpha blocking agent.

In a preferred embodiment, the TNF-alpha blocking agent is an anti-TNF-alpha monoclonal antibody such as infliximab (Remicade®), adalimumab (Humira®), certolizumab pegol (Cimzia®), and golimumab (Simponi®).

In another preferred embodiment, the TNF-alpha blocking agent is a soluble form of a TNF-alpha receptor such as etanercept (Enbrel®) (a recombinant fusion protein consisting of two soluble TNF-alpha receptors joined by the Fc fragment of a human IgG1 molecule). A pegylated soluble TNF type 1 receptor can also be used as a TNF blocking agent.

In one embodiment, the biological sample is a blood sample.

The invention also relates to a method of treating rheumatoid arthritis in a patient comprising the steps of:
a) providing a biological sample from a patient suffering from rheumatoid arthritis;
b) measuring the level of soluble VE-cadherin in the biological sample obtained at step a);
c) administering to the patient an effective amount of a TNF-alpha blocking agent;
d) providing a biological sample from the treated patient at step c);
e) measuring the level of soluble VE-cadherin in the biological sample obtained at step d);
f) comparing the levels of soluble VE-cadherin measured in biological samples at step b) and d), and

if the level of soluble VE-cadherin measured at step d) is lower that the level of soluble VE-measured at step b),

treating the patient by pursuing to administer an effective amount of a TNF-alpha blocking agent until a basal level of soluble VE-cadherin is reached.

According to the invention, one might expect to see a decrease in the level of soluble VE-cadherin in a biological sample over time during the course of effective therapy. Further, in some embodiments, multiple determinations of the level of soluble VE-cadherin over time can be made to facilitate monitoring of the treatment.

### Determination of Expression Level

Determination of the expression level of polypeptide can be performed by a variety of techniques. Generally, the expression level as determined is a relative expression level.

More preferably, the determination comprises contacting the biological sample with selective reagents such as a binding partner (e.g. an antibody), and thereby detecting the presence, or measuring the amount, of polypeptide of interest originally in the biological sample (i.e. the soluble VE-cadherin). Contacting may be performed in any suitable device, such as a plate, microtiter dish, test tube, well, glass, column, and so forth. In specific embodiments, the contacting is performed on a substrate coated with the reagent. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as an antibody-antigen complex, to be formed between the reagent and the polypeptide of interest.

Such methods comprise contacting a biological sample with a binding partner capable of selectively interacting with a biomarker protein present in the sample. The binding partner is generally an antibody that may be polyclonal or monoclonal, preferably monoclonal.

According to the present invention, “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments or derivatives. Antibody fragments include but are not limited to Fv, Fab, F(ab′)2, Fab′, dsFv, scFv, sc(Fv)2 and diabodies.

Said detection of soluble VE-cadherin can be achieved with any antibody that can binds to the extracellular region of human VE-cadherin, without binding to any other human serum protein. Preferably, said extracellular region is a region of human VE-cadherin, which comprises at least one of the EC1, EC2, EC3, EC4, EC5 extracellular domains, more preferably a region of human VE-cadherin, which comprises at least one of EC3, EC4, most preferably a region of human VE-cadherin, which comprises both EC3 and EC4. Most preferably, said anti-VEcadherin antibody is a monoclonal antibody, such as e.g., the BV9 mAb, which binds to a human VE-cadherin region comprising EC3 and EC4. The BV9 mAb is commercially available, e.g., from Abeam, 24, rue Louis Blanc, 75010 Paris, France, or from Abeam pic, UK, or from Abeam Inc., USA, under product reference ab7047.

The presence of the protein can be detected using standard electrophoretic and immunodiagnostic techniques, including immunoassays such as competition, direct reaction, or sandwich type assays. Such assays include, but are not limited to, Western blots;

agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with an antibody against the protein to be tested. A biological sample containing or suspected of containing the biomarker protein is then added to the coated wells. After a period of incubation sufficient to allow the formation of antibody-antigen complexes, the plate (s) can be washed to remove unbound moieties and a detectably labeled secondary binding molecule added. The secondary binding molecule is allowed to react with any captured sample biomarker protein, the plate washed and the presence of the secondary binding molecule detected using methods well known in the art.

Alternatively, binding agents other than antibodies may be used for the purpose of the invention. These may be for instance aptamers, which are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

In one embodiment, the method further comprises a step of dilution of the biological sample in one surfactant-containing solution before contacting said biological sample with a binding partner capable of selectively interacting with the soluble VE-cadherin.

Examples of dilution of a biological sample in a surfactant-containing solution are extensively described in the international patent application WO 2008/062314. Typically, the biological sample (e.g. a serum sample) is diluted in a solution containing a non-ionic surfactant of the Triton(R) X-100 series, such as Triton(R) X-100.

The invention will be further illustrated by the following figures and examples. However, these examples and figures should not be interpreted in any way as limiting the scope of the present invention.

### FIGURES

FIG. 1: Kinetic and dose-response experiments of VE-cadherin extracellular fragment cleavage upon TNFα challenge. A. Kinetic experiment either with PBS or TNFα (100 ng/ml) for 0 up to 60 min. The conditioned media were collected and analyzed as described in Material and Methods for VE-cadherin content. TNFα induced a time-dependent release of the 90 kDa fragment of VE-cadherin (VE-90), B. The relative amounts of VE-cadherin extracellular fragment in panel A were measured by densitometry of autoradiographs using the NIH Image J software program, C. Dose-response experiment with increasing concentrations of TNFα (from 0 up to 10 ng/ml, physiological-like conditions) for 45 min. The analysis of conditioned media was performed as indicated above, D. Quantification of the dose-response experiment. Error bars in graphs indicate S.D. The results are representative of three experiments.

FIG. 2: Implication of tyrosine kinase activities in TNFα-induced VE-cadherin cleavage. HUVECs pretreated with DMSO or genistein (50 μM) for 15 min, were then treated or not with TNFα (100 ng/ml) for 45 min. The conditioned media were collected and analyzed as described in Material and Methods for VE-cadherin content (A), The relative amounts of extracellular VE-cadherin were measured by densitometry of autoradiographs using the NIH Image J software program (B). Error bars indicate sd. The TNFα-treated cell lysates were prepared as described in the Materials and methods section and 200 μg of protein was immunoprecipitated with antibody specific to VE-cadherin C-terminus domain. The immunoprecipitates were analyzed by western blotting with phosphotyrosine antibody to (C. upper panel). The membrane was then stripped and reprobed with antibody specific to VE-cadherin (C. lower panel). Arrows on the right indicate the position of the immunoprecipitated tyrosine phosphorylated form of VE-cadherin at 125 kDa. The results are representative of at least three independent experiments.

FIG. 3: Involvement of Src family kinase(s) in VE-cadherin cleavage upon TNFα stimulation. A. HUVECs pre-treated with vehicle DMSO (0.1%), PP2 (10 μM) or genistein (50 μM) and treated with TNFα (100 ng/ml) for 45 min. The conditioned media were collected and analysed by western blotting with VE-cadherin extracellular fragment specific antibody. B. The relative amounts of extracellular VE-cadherin were measured by densitometry of autoradiographs using the NIH ImageJ software program. Error bars indicate S.D. This experiment is representative of 3 independent experiments. C. 80% confluent HUVECs were transfected with c-src targeted small interfering RNA (siSrc) or control siRNA (siCTL). After 48 h, cells were pretreated with γ-secretase inhibitor L685 (10 μM) for 15 min and treated for 45 min with TNFα in presence or absence of genistein as indicated. The treated cells media were analysed with specific antibody to detect VE-cadherin extracellular cleaved fragment. D. Immunobloting with c-src specific antibody on cell extracts 48h after transfection showed an efficient knockdown of Src kinase. This experiment is representative of 3 independent experiments.

FIG. 4: Src implication in VE-cadherin cytoplasmic tail generation upon TNFα-treatment. A. HUVECs were pretreated with the γ-secretase inhibitor L685 (5 μM) for 15 min, before incubation or not with TNFα (100 ng/ml) in the presence of DMSO (0.1%), Genistein (50 μM) or PP2 (10 μM) for 45 min. Endothelial cell extracts (10 μg) were analyzed to detect VE-cadherin cytoplasmic fragment (VE-cyto, ˜35 kDa), and the full length protein 125 kDa. Identical gel loading was attested by β-actin immunoblot. B. The proteins were analyzed as described above. c-Src specific siRNA-transfected HUVECs were treated or not with TNFα, in presence/absence of genistein (50 μM). This experiment is representative of 3 independent experiments.

FIG. 5: Requirement of metalloprotease activities for cellular conversion of 125 kDa VE-cadherin to 90 kDa and 35 kDa proteins upon TNFα challenge. A. HUVECs were serum-starved overnight, pre-treated with L685 (10 μM) for 15 min and treated for 45 min with increasing concentrations of APMA. Treated cell media were analyzed for VE-90 release as described in FIG. 1. B. The corresponding cell extracts were analyzed by western blotting with for VE-cyto. Blot stripping and reprobing with anti-β-actin was used as a control of gel loading. C. Serum-starved HUVECs were pretreated with L685 (10 μM), vehicle (DMSO, 0.1%) or GM6001 (10 μM) for 15 min prior TNFα challenge. Cell media were then collected and analysed for VE-90. D. The corresponding cell extracts (10 mg) were analysed by western blotting for VE 125 kDa and VE-cyto. Blot stripping and reprobing with anti-β-actin was used as a control of gel loading. Arrow on the right indicates the position of VE-cad 125 kDa and VE-cyto. This experiment is representative of 3 independent experiments.

### EXAMPLE

Material & Methods

Reagents: Recombinant human Tumor Necrosis Factor alpha (TNFα) was obtained from Invitrogen (USA). Tyrosine kinase inhibitor genistein (Molekula, UK), Src family kinase inhibitor PP2 (Calbiochem, CA), were used in this study. Sodium orthovanadate and H₂O₂, matrix metalloproteinase inhibitor GM6001, matrix metalloproteinase activator amino-phenyl-mercuric acetate (APMA), were purchased from Sigma Aldrich. Commercially available antibodies were purchased from different sources: goat polyclonal anti-VE-cadherin cytoplamic domain (C19) and mouse monoclonal anti-VE-cadherin extracellular fragment (clone BV9) from Santa Cruz Biotechnology (Santa Cruz, USA), mouse monoclonal anti-phosphotyrosine 4G10 (Millipore), mouse polyclonal anti-β-actin (Sigma Aldrich, CA, USA), rabbit polyclonal anti-c-Src (Invitrogen, USA) Horseradish peroxidase-conjugated purified rabbit anti-mouse IgG was from Bio-Rad (Hercules, CA, USA). Rabbit polyclonal anti-MMP2 precursor was purchased from (Epitomics, France). Enhanced chemiluminescence detection reagents were purchased from Perkin-Elmer (Courtaboeuf, France). Nitrocellulose was obtained from Schleicher and Schuell (Ecquevilly, France). The micro-bicinchoninic acid protein assay reagent kit was from Fischer-Scientific (France).

Buffers: Buffer A was: 20 mM Tris/acetate (pH 7.0), 0.27 M sucrose, 1% (v\v) Triton X-100, 1 mM dithiothreitol (DTT), 1 mM EDTA, 1 mM EGTA, 1 mM Na3VO4, 1 mM benzamidine, 4 μg\ml leupeptin and 1 μg/ml pepstatin A. Buffer B was: 10 mM Tris/HCl (pH 7.4), 150 mM NaCl, 1 mM EDTA, 1 mM EGTA, 1% (v/v) Triton X-100 and 0.5% (v/v) Nonidet P-40, BSA 0.1%. Sodium pervavadate solution: A stock solution of pervanadate at 50 mM was prepared by mixing equal volumes of 0.1 M sodium orthovanadate and 0.2 M H2O2. After incubation at room temperature for 20 min, the solution was diluted in phosphate-buffered saline (PBS) to a final concentration of 5 mM before use. The final concentration used for cell treatment was 5 μM.

Cell culture, extraction and immunoprecipitation: Endothelial cells were obtained from human umbilical vein (HUVECs) and grown to reach confluence in M199 medium supplemented with 10% fetal bovine serum (FCS), 2% Low Serum Growth Supplement LSGS (Cascade Biologics, USA) as previously described (Garnier-Raveaud et al., 2001). The 80% confluent HUVECs (passages 2 or 4) were used for most experiments. Confluent cells in 60 mm dishes were serum-starved in 1% fetal calf serum in M 199 for 24 h prior to testing. Before TNFα stimulation the cells were serum-starved for 2 hours, and then pre-treated for 15 min with sodium pervanadate (10 μM) and/or inhibitors as mentioned in text and figure legends. HUVECs were then stimulated with different concentrations of TNFα for various lengths of time as indicated. Otherwise, in most parts of this study, precisely in inhibition experiments TNFα was used at high concentration (i.e 100 ng/mL). As the inhibitors were diluted in dimethyl-sulfoxide (DMSO), control cells were systematically treated with DMSO 0.1% in any inhibition experiment. The use of DMSO did not have any effect on phosphorylation and proteolysis processes of VE-cadherin.

Cells stimulation was stopped by medium collection and addition of 500 μl of ice-cold buffer A. The cells were harvested, homogenized and sonicated for 20 seconds. Cell lysates were collected after centrifugation for 15 min at 40° C. at 14,000 g. The protein concentration was measured using the micro-bicinchoninic acid protein assay and cell lysates were stored at −80° C. before use.

For conditioned medium analysis, the medium was collected and centrifuged for 15 minutes at 14,000 g at 4° C. The resulting supernatant was collected and proteins were concentrated by trichloro acetic acid precipitation (10%) overnight. After centrifugation at 14,000 g for 30 min, the pellet was re-suspended in Tris 50 mM (pH 11) and analyzed by SDS-PAGE and Western blotting.

For immunoprecipitation, 500 μg of protein of different cell extracts were incubated with 2 μg of the monoclonal antibody to VE-cadherin in 500 μl of buffer B for 1 h at 4° C. Immunoprecipitations were performed with protein G-Sepharose beads and fractions were collected for 45 min at 4° C. The fractions were centrifuged for 5 min at 4° C. and the immunoprecipitates were washed five times with buffer B. Samples were eluted by boiling in with Laemmli buffer containing a final concentration of 2.5% (v/v) β-mercaptoethanol, and subjected to SDS-PAGE (12% Acrylamide, 0.2% bis-acrylamide).

Src small interfering RNA (siRNA) transfection in HUVECs: A pool of 4 target-specific small interfering RNA (siRNAs) designed to target c-Src (Santa Cruz Biotech, sc-29228) and the appropriate control siRNA purchased from Invitrogen (45-2001). The transfection protocol used was from Qiagen using HiPerFect Transfection Reagent. HUVECs were grown in 60 mm dishes in M199 with 10% FCS to reach 80% confluency. Before transfection, culture medium was replaced by M199 only for 3 hours. The c-Src and control siRNAs (30 nm) were preincubated with transfection reagent for 10 min (20 μL) (Qiagen, Valencia) according to the manufacturer, and the transfection mixture was added to the cells (20 μL/mL) for 3 hours in the absence of serum. The cells were used 48 hours after transfection.

Western blotting: Proteins were resolved by SDS-PAGE and transferred to nitrocellulose. The blots were saturated with non-fat milk, incubated with the specific primary antibody followed by the specific horseradish-peroxidase-conjugated secondary antibody and revealed by chemiluminescence.

Data analysis: All of the experiments were repeated at least three times. Values represent the mean±sd of three determinations from three different wells or dishes in the same experiment. Each experiment was performed at least three times under identical or similar conditions with comparable results.

Rheumatoid arthritis patients: Our analysis was based on 63 patients from the Very Early Rheumatoid Arthritis (VErA) cohort (Goeb et al., 2005 & Goeb et al., 2008) who had peripheral rheumatisms characterized by the swelling of ≧2 joints, lasting ≧4 weeks and evolving for <6 months, and had not received any systemic Disease-modifying antirheumatic drugs (DMARDs) or glucocorticoids. The patients were recruited prospectively in the Rheumatology Department of the Rouen University Hospital. They had a standardized follow up. Patients' clinical, biological and radiological parameters were recorded at inclusion, then every 6 months. Herein, only inclusion data were considered. The clinical parameters recorded were demographic information (age, sex), rheumatism duration (defined as the date the first symptoms appeared), joint-pain intensity evaluated with a visual analog scale (range: 0-100 mm), and the numbers of painful and swollen joints among the 44 examined. Among the biological parameters of these patients evaluated, the following were retained: Erythrocyte Sedimentation Rate ESR (mm/1^{st} h); C-reactive protein CRP (mg/l); autoantibodies: Rheumatoid factors RFs detected by agglutination (latex-fixation and Waaler-Rose) and anti-cyclic citrullinated protein anti-CCP2 detected with a commercialized kit (Euroimmun®, Groβ Grönau, Germany). For the first 2 years, treating rheumatologists were given recommendations so that the included patients would be treated homogeneously. The guidelines had been devised before early intensive DMARDs administration became the internationally accepted strategy and prior to biotherapy availability in France. Schematically, it was recommended not to use systemic glucocorticoids unless necessitated by very active disease, and then briefly at the lowest possible dose. For DMARDs, it was recommended to start with hydroxychloroquine (6 mg/kg/day), to be replaced or combined with oral methotrexate, starting at 7.5 mg/week.

Statistical analysis for RA cohort: In addition to a descriptive analysis of the studied population, the inventors used the Spearman correlation coefficient to evaluate the relation between the titers of soluble serum VE-cadherin and clinical or biological parameters collected at baseline prior to initiation of DMARDs and/or corticosteroids and during the first year of follow-up. All western blot bands have been subjected to densitometry and data are expressed in arbitrary units as the mean±sd of at least three identical experiments and subjected to student test. For all tests, P≦0.05 was considered as significant.

Results

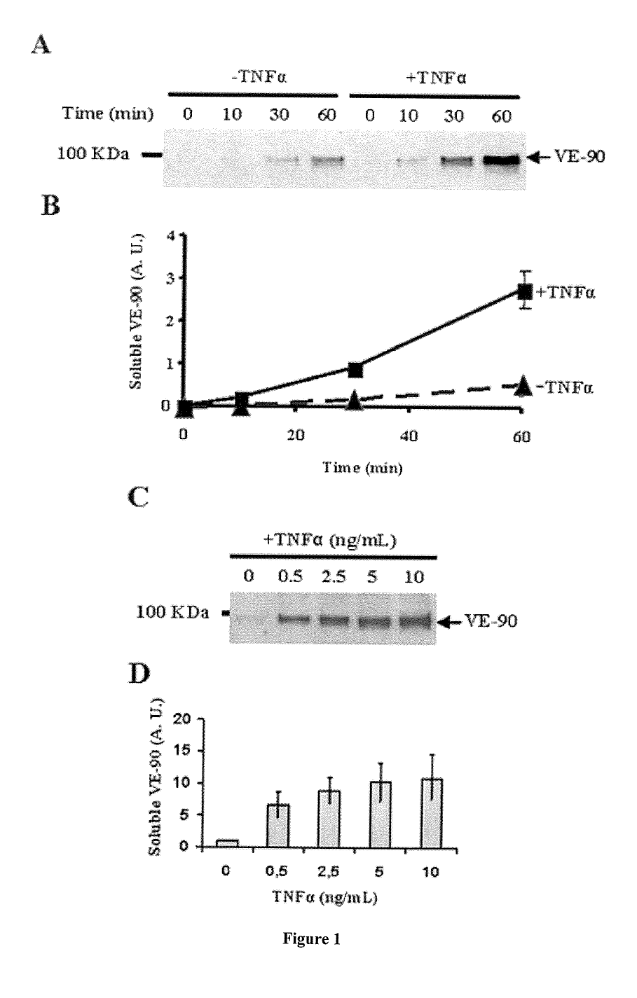

TNFα induces post-translational processing of 125kDa VE-cadherin and generates its 90 kDa extracellular domain: Early in vitro and in vivo studies have demonstrated the role of TNFα in causing endothelial cell monolayer disruption leading to increased permeability. Because VE-cadherin extracellular domain is responsible for the strong endothelial cell-cell adhesion, the inventors examined whether it was rapidly processed upon TNFα challenge. HUVECs were treated or not for 5 to 60 min with TNFα at 100 ng/ml, a concentration reported to induce a rapid VE-cadherin tyrosine phosphorylation and an increased endothelial cell permeability. The respective conditioned media were analyzed by Western blot with monoclonal antibody to VE-cadherin. FIG. 1A shows the presence of one immunoreactive band with an apparent molecular weight of 90 kDa. Based on its size and immunoreactivity, it corresponds to the full length of the extracellular domain of VE-cadherin (VE-90) (Lambeng et al., 2005). The VE-90 was rapidly detected after 10 min of TNFα stimulation and increased in a time-dependent manner up to 60 min, while it was barely detectable in untreated cells. Quantification of the immunoreactive bands using ImageJ software is illustrated in FIG. 1B. This result demonstrates that TNFα is a potent inducer of VE-cadherin extracellular domain cleavage. A dose-response curve was then performed using TNFα concentrations related to pathophysiology. The result illustrated in FIG. 1C shows a strong effect of TNFα early detected at a low concentration of 0.5 ng/mL, which increased in a dose-dependent manner to reach a plateau between 5 to 10 ng/ml (FIG. 1D). Altogether, these results demonstrate that TNFα is a powerful inducer of VE-cadherin extracellular domain cleavage at concentrations that correlate with pathophysiological conditions.

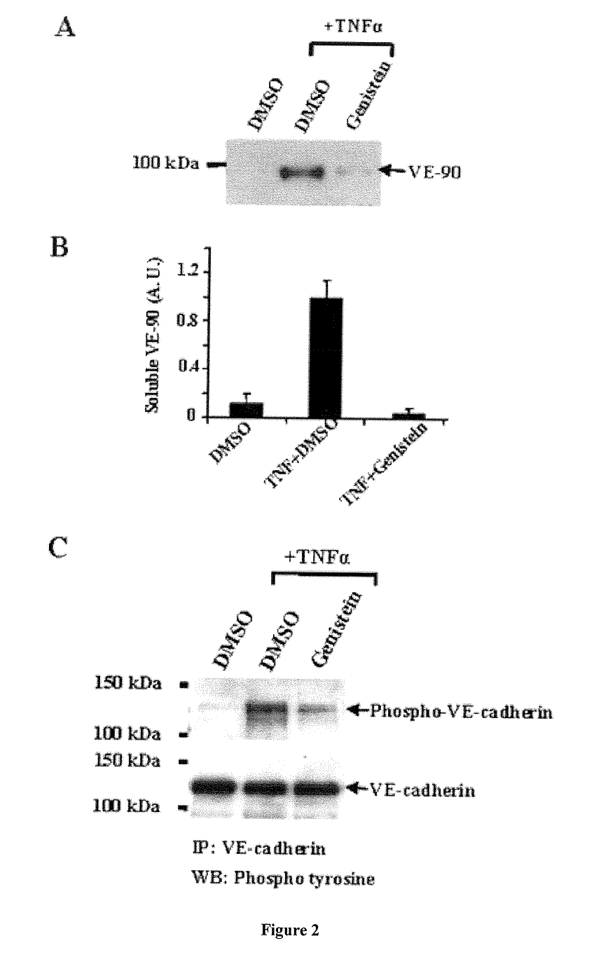

Tyrosine kinases are required for TNFα-induced VE-cadherin cleavage: Adherens junction assembly is known to be regulated, in part, through tyrosine phosphorylation of proteins within the multiprotein complex, including α-catenin, β-catenin, p120-catenin and VE-cadherin. In order to examine whether tyrosine kinases were involved in the observed TNFα-induced VE-cadherin cleavage, the same experiment as described above was performed using genistein (50 μM), a broad-spectrum tyrosine kinase inhibitor, prior to TNFα challenge. Analysis of VE-90 in the conditioned media showed that genistein strongly decreased TNFα-induced VE-cadherin extracellular domain cleavage (FIGS. 2A, B). In addition, genistein inhibited TNFα-induced VE-cadherin tyrosine phosphorylation (FIG. 2C) without affecting cell viability. This data demonstrates that tyrosine kinases are required for TNFα-induced VE-cadherin cleavage.

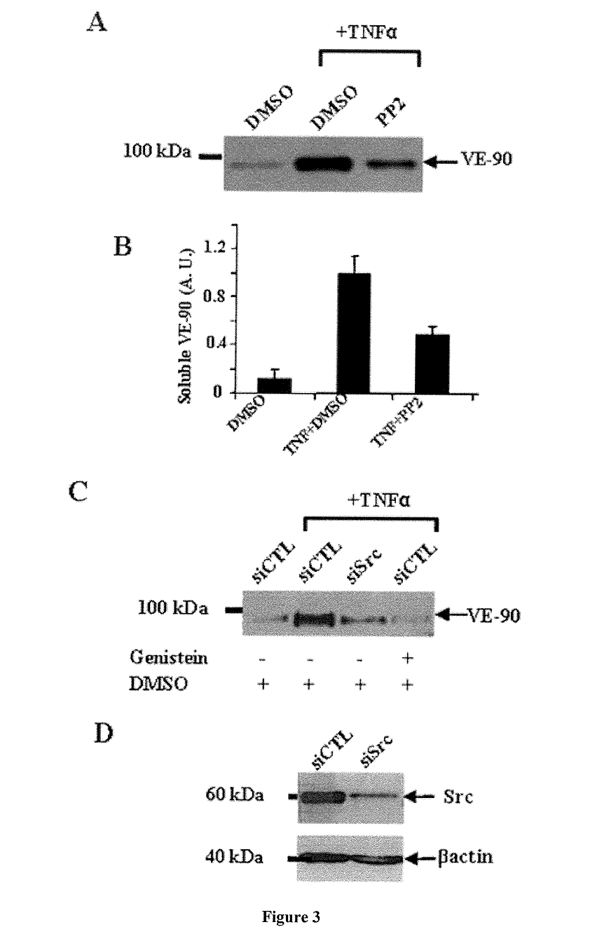

TNFα-induced VE-cadherin cleavage is dependent upon Src kinase activity: Given the large body of evidence supporting the major role for Src kinase in disassembly of adherens junctions in endothelial cells and VE-cadherin phosphorylation, the role for Src family kinases in TNFα-induced VE-cadherin cleavage was further determined. To that exent, HUVECs were pre-treated with the Src family kinase inhibitor, PP2 prior to TNFα stimulation. As shown in FIGS. 3A, B, the strong TNFα-induced VE-90 release observed after 45 min was strongly decreased by PP2 treatment without affecting cell viability. In addition, the knock down of Src using Src specific siRNA efficiently impaired TNFα-induced VE-90 release in cell medium but not with control siRNA (FIGS. 3C, D). This experiment strongly confirmed the specific requirement of Src kinase activity for TNFα-induced VE-90 release.

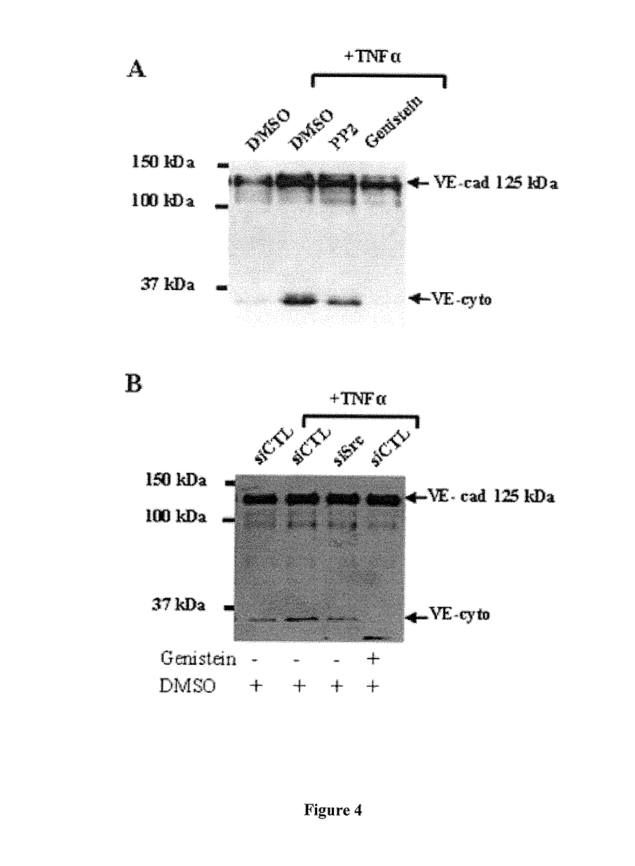

VE-90 release is associated with the generation of VE-cadherin cytoplasmic domain: The inventors further examined whether the cleavage of VE-cadherin extracellular domain induced by TNFα was concomitantly associated with the generation of its cytoplasmic domain (VE-cyto). This process was analyzed in endothelial cell extracts after TNFα stimulation, in the presence of the γ-secretase inhibitor L685 (10 μM), to prevent further proteolytic degradation of the VE-cadherin cytoplasmic domain. Under these conditions, the appearance of VE-cyto was clearly detected upon TNFα challenge while it was not detected in untreated cells (FIG. 4). In addition, the VE-cyto generation was strongly impaired by a pre-treatment with either with PP2 (10 μM), or genistein (50 μM), or Src-specific siRNA (FIGS. 4A, B). These data which further confirm the role for Src kinase in TNF-induced VE-cadherin extracellular domain cleavage-and VE-cyto generation.

The balance between the activities of protein tyrosine kinases and phosphotyrosine phosphatases (PTPases) determines the level of tyrosine phosphorylation. To further confirm the importance of tyrosine phosphorylation processes in TNFα-induced VE-cadherin cleavage, the next experiment was performed in HUVECs pretreated or not by sodium pervanadate (Na3VO4), a strong tyrosine phosphatase inhibitor. Importantly, we demonstrate that tyrosine phosphatase blockade led to a tremendous increase in VE-cyto generation upon cytokine treatment as compared to non pre-treated cells. Genistein treatment still decreased the generation of VE-cyto in both conditions. This result demonstrates that inhibition of tyrosine phosphatase activities facilitates VE-cadherin cleavage and further confirms the involvement of tyrosine phosphorylation processes in TNFα-induced VE-cadherin cleavage.

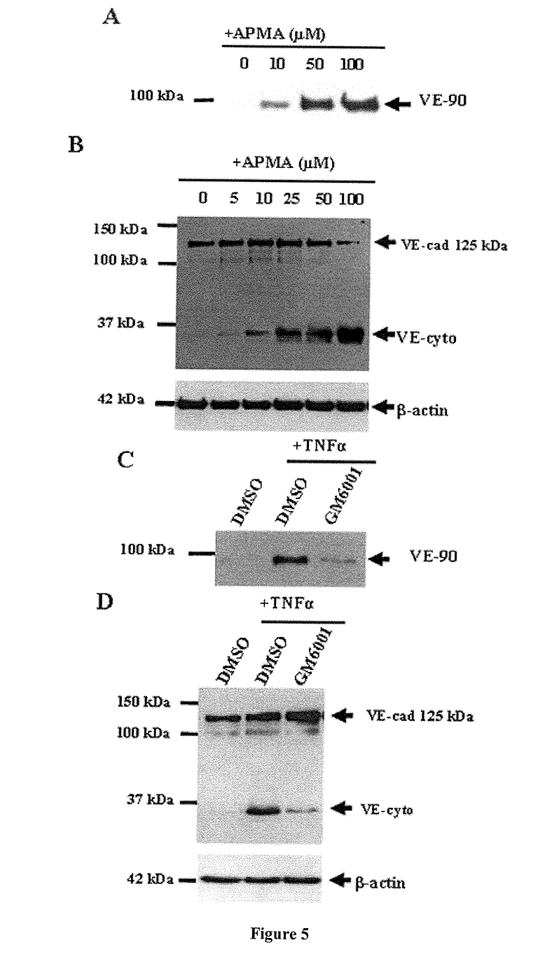

Metalloproteases activities (MMPs) are required for cellular conversion of 125 kDa VE-cadherin to 90 kDa and 35 kDa proteins upon TNFα challenge: A large body of evidence have shown that MMPs are involved in joint destruction in RA and are associated with endothelial dysfunction. The inventors thus examined whether MMPs could target VE-cadherin. To that purpose, cell medium was analyzed for the presence of VE-90 after a dose-response experiment performed using amino-phenyl mercuric acetate (APMA), a broad-spectrum activator of MMPs. As shown in FIG. 5A, increasing concentrations of APMA from 10 to 100 μM, induced a dose-dependent VE-90 release in HUVEC media. In FIG. 5B, analysis of the corresponding cellular extracts demonstrated that APMA-treatment induced a dose-dependent decrease in the 125 kDa full length protein and concomitantly a strong increase in the VE-cyto generation. This result confirmed that VE-cadherin is a substrate for MMPs which generate the same 90 kDa fragment released in HUVEC medium as seen upon TNFα stimulation.

The involvement of MMPs in TNFα-induced VE-cadherin cleavage was further confirmed by the use of GM6001, a broad-spectrum inhibitor of MMPs. HUVECs were treated for 15 min with GM6001 (10 μM) prior to TNFα stimulation. Cell lysates and conditioned media were analyzed for the presence of VE-cyto and VE-90 respectively. As shown in FIGS. 5C, D, the MMPs inhibitor drastically decreased TNFα-induced VE-90 release in cell media and almost completely abrogated the VE-cyto generation, without affecting cell viability. Several MMPs such as MMP2, MMP9, ADAM-10 have been involved in VE-cadherin cleavage. The MMPs are initially synthesized as inactive zymogens with a pro-peptide domain that should be removed to render the enzyme active. The inventors further examined the presence of MMPs (pro-enzyme and enzymes) in conditioned media from AMPA-treated HUVECs. This analysis was performed using specific antibodies for each species. ProMMP2 (precursor form) was detected (72 kDa) in a time and dose dependent manner upon APMA challenge whereas MMP9 (92 kDa) was not detectable at any time tested. The proMMP-2 maturation was correlated to the time-dependent VE-90 release as its pro-enzyme decreased according APMA concentration. The detection of active MMP2 was confirmed by a zymography experiment showing a time-dependent relationship between VE-90 generation and proMMP2 maturation into active MMP-2. Both ADAM-10 precursor (85 kDa) and active forms (60 kDa) were detectable in cell lysates but no differences in the ADAM-10 precursor/active balance were found upon APMA treatment. Altogether, these results strongly suggest the requirement of MMPs in TNFα-induced VE-90 generation and more specifically the involvement of MMP2 activity in VE-90 release.

Soluble 90 kDa VE-cadherin is present in RA patient sera and correlates with disease activity scores (DAS44): Because TNFα is the major pro-inflammatory cytokine involved in RA, the inventors next determined whether RA patients exhibited a truncated form of VE-cadherin that could be detected in their sera. Sixty three patients from a RA population (Goeb et al., 2008), DMARD and corticosteroids naïve, whose baseline characteristics are summarized in Table 1, were studied. Diluted patient sera were analyzed by SDS-PAGE and western blotting with an anti-VE-cadherin antibody directed against its extracellular domain. One immunoreactive band of 90 kDa was detected in all RA patient sera. Based on the immunoreactivity and its molecular weight, the inventors concluded that this fragment corresponded to the VE-90 observed in HUVECs conditioned media upon TNFα treatment. VE-90 immunoreative bands detected in all sera were quantified by densitometry using ImageJ NIH Software. The data were analyzed in order to know whether titers of soluble VE-cadherin were related to disease activity. In this respect, we found a relationship between this marker and DAS44 (Disease Activity Score computed on 44 joints) reflecting global disease activity, both at baseline (r=0.35; p=0.007) and over the first year of follow-up when considering their mean values (r=0.29; p=0.02). This correlation was not due to traditional parameters of inflammation since no link was observed between soluble VE-cadherin and ESR or CRP. Furthermore, no relationship was shown between soluble VE-90 and markers of autoimmunity (RF and anti-CCP2) or osteoarticular destruction (Sharp scores) measured at baseline. These results demonstrate for the first time the occurrence of VE-90 release in RA disease.

TABLE 1
Demographic, clinical, biological and radiographic characteristics
of the 63 patients included in the present study.

| Age, years | 53 | (26-76) |
|---|---|---|
| F/M sex ratio | 1.86 | |
| Disease activity score 44 | 2.95 | (1.31-7.21) |
| ESR, mm/hour | 12 | (2-110) |
| CRP level, mg/l (N < 5 mg/l) | 6 | (5-185) |
| RF positivity (%) | 34 | |
| Anti-CCP2 positivity (%) | 27 | |
| RF levels (Latex fixation test) levels, IU/ml | 0 | [0-938] |
| (N < 20 IU/ml) | | |
| Anti-CCP2 autoantibodies, AU (N < 10 AU) | 0 | (0-100) |
| Modified-Sharp scores | | |
| Erosion | 0 | (0-6) |
| Total | 0 | (0-16) |
| VE-cadherin, AU | 37 | (6-100) |

Values are expressed as medians (range), unless indicated otherwise
ESR = erythrocyte sedimentation rate at 1st hours;
CRP = C-reactive protein;
N = normal value;
RF = rheumatoid factor;
AU = arbitrary units:
CCP2 = cyclic citrullinated protein.

Discussion

Patients with RA have an increased morbidity and mortality due to cardiovascular disease (CVD). Traditional cardiovascular risk factors cannot fully explain the increase but inflammation has been shown to contribute to the increased CVD in RA patients.

Inflammation leads to endothelial dysfunction, a sign of very early atherosclerosis, which can be assessed by impaired-endothelial flow-mediated vascular dilatation of peripheral arteries, measured by ultrasonography. Another potential way to examine endothelial dysfunction in RA might be to examine whether the most targeted cytokine in the disease TNFα, could affect the adhesive properties of a major component of endothelial adherens junctions: VE-cadherin.

The above data demonstrate that TNFα induced a dose-dependent release of VE-cadherin extracellular domain and a decrease of the full-length protein expression on the cell surface. This observation of great potential interest suggests that VE-cadherin adhesive properties appeared to be regulated by TNFα, and might contribute to the reported TNFα-induced endothelial permeability. A wide group of transmembrane proteins, including adhesion molecules, TNFα receptor, transforming growth factor-α (TGF-α), angiotensin-converting enzyme, β-amyloid precursor protein, and syndecan, undergo ectodomain cleavage. In numerous cases, this process is activated by the phorbol 12-myristate 13-acetate

(PMA), a well known activator of the protein kinase C (PKC), indicating the involvement of phosphorylation as an important regulatory mechanism of these proteins cleavage. VE-cadherin tyrosine phosphorylation is a mechanism involved in endothelial cell-cell dissociation and increased permeability. While the role of tyrosine kinases has been largely established in this process, the precise molecular mechanisms remain elusive. Of interest, in this study, we demonstrate the implication of tyrosine kinases such as Src family kinases, in TNFα-induced VE-cadherin cleavage. Increasing Src kinase activity has been associated with VE-cadherin phosphorylation, adherens junction dissociation and endothelial permeability augmentation. Thus Src activity can also be involved in the reported TNFα-induced endothelial permeability through VE-cadherin cleavage. Another interesting point is that the observed time-dependent and dose-dependent release of VE-90 upon TNFα was delayed as compared to the reported time-dependent phosphorylation of the protein. In addition, results obtained using tyrosine kinases and phosphatases inhibitors demonstrated the correlation between both events. These observations strongly support the hypothesis that VE-cadherin tyrosine phosphorylation in its cytoplasmic domain is an early event which precedes the cleavage of its extracellular domain. Data are in agreement with two previous studies reporting independently VE-cadherin tyrosine phosphorylation and cleavage of its extracellular fragment cleavage upon thrombin stimulation.

Several evidences suggest that proteolytic cleavage of cell surface proteins, i.e. ectodomain shedding, appears to be mediated by members of the metzincin superfamily of zinc-dependent proteases that include the matrix metalloproteinases (MMPs) and ADAMs (A Disintegrin And Metalloproteinase). The MMPs and adamalysins are considered to be major mediators of cartilage destruction in RA. Interestingly, MMP-2, 7 and 9 were the most documented to induce VE-cadherin shedding in several circumstances, namely apoptosis, diabetic retinopathy and Dengue virus infection. In addition, adamalysins such as ADAM-9 and 10 have been shown respectively to mediate VE-cadherin cleavage during retinal neovascularization and in HUVECs upon thrombin stimulation respectively. These observations are in agreement with the present results and support the hypothesis that TNFα-induced VE-cadherin cleavage could be mediated by several of these proteases. Further investigations are ongoing to determine more precisely whether one of thesesproteases is specifically involved in VE-cadherin cleavage upon TNFα stimulation. Preliminary data suggest a potential role of MMP-2 activity in this process. In addition, the regulation of its catalytic activity through tyrosine phosphorylation is another question to be addressed. Because VE-cadherin extracellular domain is of major importance for cell-cell adhesiveness, it remains to be seen whether the VE-90 release is a general process causing endothelial permeability in inflammation and edema.

Elevated soluble VE-cadherin has been reported to be associated to diabetic retinopathy and coronary atherosclerosis. However, the molecular weight of the soluble protein was not documented and the mechanisms involved in this process were not studied. Despite its predominant role as a gatekeeper for neutrophil transmigration, VE-cadherin has never been studied in RA disease. The current study is the first to demonstrate the presence of soluble VE-cadherin in 63 RA patients and to investigate its potential clinical interest in RA disease. This observation is in agreement with the present in vitro experiments on HUVECs stimulated with TNFα. It remains to be examined whether the early effect of TNFα on VE-90 release may be related to the long range effect of this cytokine in RA. Preliminary data have highlighted a correlation between soluble VE-cadherin levels in early RA patient sera and their disease activity scores. Actually, DAS is one of the most important tools for evaluating RA disease activity and the responsiveness of each patient to therapy. Interestingly, this relationship seems independent of CRP levels, suggesting that VE-90 could constitute a new marker of disease activity follow-up, particularly in the subset of RA patients with no CRP increase.

A routine biological assay for the detection and quantification of soluble VE-cadherin in RA patients' sera is further required. The development of easily applicable diagnostic-type assay like Enzyme-linked Immuno-sorbent Assay would be of major interest. To that purpose the sequencing of the VE-90 from RA patient sera is underway to define the best strategy to produce the same fragment in human cell that will be used to develop an internal standard curve to determine precisely the concentration of VE-90 in patients' sera. Therefore further clinical trials will be more feasible and of major importance to determine whether VE-90 will be interesting for prediction of prognosis and/or drug responsiveness, especially for new therapies in RA including TNFα inhibitors (Infliximab, Adalimumab, Etanercept, Certolizumb pegol).

References:

Throughout this application, various references describe the state of the art to which this invention pertains. The disclosures of these references are hereby incorporated by reference into the present disclosure.

Corada M, Liao F, Lindgren M, Lampugnani M G, Breviario F, Frank R, Muller W A, Hicklin D J, Bohlen P, Dejana E. Monoclonal antibodies directed to different regions of vascular endothelial cadherin extracellular domain affect adhesion and clustering of the protein and modulate endothelial permeability. Blood. 2001; 97(6):1679-84.

Garnier-Raveaud S, Usson Y, Cand F, Robert-Nicoud M, Verdetti J, Faury G. Identification of membrane calcium channels essential for cytoplasmic and nuclear calcium elevations induced by vascular endothelial growth factor in human endothelial cells. Growth Factors. 2001; 19(1):35-48.

Goeb V, Dieude P, Daveau R, Thomas-L'otellier M, Jouen F, Hau F, et al. Contribution of PTPN22 1858T, TNFRII 196R and HLA-shared epitope alleles with rheumatoid factor and anti-citrullinated protein antibodies to very early rheumatoid arthritis diagnosis. Rheumatology (Oxford). 2008 August; 47(8):1208-12.

Goeb V, Dieude P, Vittecoq O, Mejjad O, Menard J F, Thomas M, et al. Association between the TNFRII 196R allele and diagnosis of rheumatoid arthritis. Arthritis Res Ther. 2005; 7(5):R1056-62.

Lambeng N, Wallez Y, Rampon C, Cand F, Christe G, Gulino-Debrac D, et al. Vascular endothelial-cadherin tyrosine phosphorylation in angiogenic and quiescent adult tissues. Circ Res. 2005 Feb. 18; 96(3):384-91.

1. A method of treating rheumatoid arthritis in a subject comprising the steps of:
a) providing a biological sample from a subject,
b) measuring the level of soluble VE-cadherin in the biological sample obtained at step a);
c) comparing said level of soluble VE-cadherin with a predetermined reference value, and
if the level of soluble VE-cadherin measured at step b) is higher that the predetermined reference value,
treating the subject until a basal level of soluble VE-cadherin is reached.

2. The method of claim 1, wherein the patient is treated with an effective amount of a disease modifying antirheumatic drugs (DMARD).

3. The method of claim 1, wherein the patient is treated with an effective amount of a TNF-alpha blocking agent.

4. The method of claim 1, wherein the subject is patient with an effective amount of an anti-TNF-alpha antibody or a soluble form of a TNF-alpha receptor.

5. The method of claim 1, wherein the biological sample is a blood sample.

6. A method of treating rheumatoid arthritis in a patient comprising the steps of:
a) providing a biological sample from a patient suffering from rheumatoid arthritis;
b) measuring the level of soluble VE-cadherin in the biological sample obtained at step a);
c) administering to the patient an effective amount of a TNF-alpha blocking agent;
d) providing a biological sample from the treated patient at step c);
e) measuring the level of soluble VE-cadherin in the biological sample obtained at step d);
f) comparing the levels of soluble VE-cadherin measured in biological samples at step b) and d), and
if the level of soluble VE-cadherin measured at step d) is lower that the level of soluble VE-measured at step b),
treating the patient by pursuing to administer an effective amount of a TNF-alpha blocking agent until a basal level of soluble VE-cadherin is reached.

7. The method of claim 6, wherein the TNF-alpha blocking agent is an anti-TNF-alpha antibody or a soluble form of a TNF-alpha receptor.
